(19) United States
(12) Patent Application Publication
(10) Pub. No.: US 2010/0192730 A1
(43) Pub. Date: Aug. 5, 2010

(54) CONTROL DEVICE FOR A TATTOO MACHINE, AND TATTOO SYSTEM

(76) Inventor: William DUBIN, Larchmont, NY (US)

Correspondence Address: KENYON & KENYON LLP, ONE BROADWAY, NEW YORK, NY, 10004, US

(21) Appl. No.: 12/698,653

(22) Filed: Feb. 2, 2010

Related U.S. Application Data
(60) Provisional application No. 61/206,771, filed on Feb. 4, 2009.

(51) Int. Cl. A61B 17/34; A61M 37/00

(52) U.S. Cl. 81/9.22; 606/169

(57) ABSTRACT

A control device for a tattoo machine, and a tattoo system. The control device adapted to control reciprocation of a needle assembly of the tattoo machine using a pulsed signal. A duty cycle of the pulsed signal controlling a stroke length of the needle assembly. A frequency of the pulsed signal controlling a stroke frequency of the needle assembly. Duty cycle and frequency may be controlled independently of one another.

### CROSS REFERENCE

The present application claims benefit under 35 U.S.C. §119 of U.S. Provisional Application No. 61/206,771, filed on Feb. 4, 2009, which is expressly incorporated herein by reference in its entirety.

### FIELD

The embodiments of the present invention relate to a control device for a tattoo machine, and more particularly, the embodiments of the present invention relate to control of a tattoo machine using a pulsed signal. In one embodiment, an astable multivibrator-triggered monostable multivibrator control device is used to allow an operator to vary the relative percentage of time within a cycle that a tattoo machine is ON and OFF. The ON state, therefore, can be made longer or shorter compared to the amount of time the tattoo machine is in the OFF state within one cycle. The embodiments of the present invention also control the frequency of the cycles per second of the tattoo machine. Controlling the interaction among the frequency of strokes, the length of time the pulse is on during each stroke, and the length of the stroke itself enables the operator to tune a tattoo machine over a wide range of frequencies.

### BACKGROUND INFORMATION

The configuration of a conventional tattoo machine 10 is shown in FIGS. 1A and 1B, which show a side view and front view, respectively (the front view being shown without a needle assembly 99). The tattoo machine 10 includes a frame 12. The frame 12 is electrically and magnetically conductive, and comprises a horizontal support portion 14 and a vertical support portion 16. The horizontal support portion 14 of the frame 12 supports first and second electromagnetic coils 18 and 20, respectively, that are series-connected and have distal ends 102 and 103, respectively.

The first and second electromagnetic coils 18 and 20 have bottom core portions, and are each coupled to the horizontal support portion 14 of the frame 12 by screws. The screws pass through the horizontal support portion 14 of the frame 12, into threaded holes in the bottom core portions of the first and second electromagnetic coils 18 and 20.

The horizontal support portion 14 of the frame 12, when coupled to the first and second electromagnetic coils 18 and 20, respectively, acts as a U-shaped magnet. The series connection of the first and second electromagnetic coils 18 and 20 is reversed on the second electromagnetic coil 20 so as to allow the distal end 102 of the first electromagnetic coil 18 to become a magnetically negative pole and the distal end 103 of the second electromagnetic coil 20 to become a positive pole, together forming a single U-shaped magnet.

The tattoo machine 10 further includes a side piece 30. The side piece 30 is flat, rectangular, electrically conductive, and has a lower portion 62 with a side, and an upper portion 32 with a side and an inside. The lower portion 62 of the side piece 30 extends out to the side thereof far enough to allow clearance of the first electromagnetic coil 18 and then extends upwardly from the horizontal support portion 14 of the support structure 12 allowing sufficient height for clearance of the first electromagnetic coil 18, an armature bar 56, a spring 26/28 having an angularly extending portion 104, a front standoff 64 having a threaded hole 106 and a leading edge 120, and a conductive contact screw 34.

The conductive contact screw 34 is held in place by being threaded through the front standoff 64, at the threaded hole 106 therein, making it adjustable in length when threaded through the threaded hole 106 in the front standoff 64 and held in place by a set screw 66.

The front standoff 64 is coupled to the upper portion 32 of the side piece 30 by a screw 72. The screw 72 is insulated from the side piece 30 by a non-conducting plastic recessed washer 70 on the outer side of the upper portion 32 of the side piece 30 and a non-conducting recessed washer 68 on the inside of the upper portion 32 of the side portion 30 preventing the screw 72 from contacting the side piece 30 and preventing the front standoff 64 from contacting the side piece 30. A solder lug 108 is placed between the leading edge 120 of the front standoff 64 and the recessed washer 68 thus insulating the conductive contact screw 34 and the front standoff 64 from the side piece 30.

The contact screw 34 is positioned through the threaded hole 106 in the front standoff 64 in a downwardly direction to allow it to contact the angularly extending portion 104 of the spring 26/28 when the spring 26/28 is in its rest position. A solder lug 108 is placed next to the leading edge 120 of the front standoff 64 and next to the inner recessed washer 68.

The screw 72 goes through the non-conducting plastic recessed washer 70, then through the upper portion 32 of the side piece 30, then through the inner recessed washer 68, then through the solder lug 108, and threads into the threaded hole 106 in the front standoff 64 until tight.

The frame 12 further includes a negative terminal 22 and a positive terminal 24 that is insulated from the frame 12 and which are for connecting to a positive output and a negative output, respectively, of a power supply.

The spring 26/28 is coupled to the frame 12, extends horizontally from the negative terminal 22 of the frame 12, and comprises the angularly extending portion 104. The angularly extending portion 104 of the spring 26/28 is either integral with or separate and secured to the spring 26/28.

The positive terminal 24 of the frame 12 is electrically coupled to the first electromagnetic coil 18. The first electromagnetic coil 18 is connected in series to the second electromagnetic coil 20 and to the conductive contact screw 34.

The tattoo machine 10 further includes a capacitor 36. The positive terminal 24 of the support structure 12 is also connected in series to the capacitor 36 and to the conductive contact screw 34, and as such, the capacitor 36 is connected in parallel to the first and the second electromagnetic coils 18 and 20, respectively. The capacitor 36 is provided to reduce or eliminate arcing.

In the rest position of the spring 26/28, a closed circuit is formed, since the conductive contact screw 34 is in contact with the angularly extending portion 104 of the spring 26/28. The spring 26/28 is electroconductively connected to the negative terminal 22 of the support structure 12.

The tattoo machine 10 further includes a cylindrical tube 38 having an upper end 40, a lower end 42, and a lumen 43, a needle bar 44 having an upper end 46 and a lower end, and a tattoo needle at the flower end of the needle bar 44. The cylindrical tube 38 is connected to the support structure 12, and the needle bar 44 is provided within the lumen 43 of the cylindrical tube 38.

The upper end 46 of the needle bar 44 extends past the upper end 40 of the cylindrical tube 38. The lower end of the needle bar 44 is positioned within the cylindrical tube 38 and is coupled within the cylindrical tube 38 to the tattoo needle. The lower end 52 of the tattoo needle (when connected to needle bar 44) extends past the lower end 42 of the cylindrical tube 38.

The tattoo machine 10 further includes an armature 56. The armature 56 is formed of a magnetic material, is connected to the spring 26/28, extends horizontally along at least a portion of the spring 26/28, and has a distal end 58. The distal end 58 of the armature 56 is connected to the upper end 46 of the needle bar 44. The armature 56 is orthogonal to the needle bar 44 when the armature 56 is in its rest position.

The operation of the tattoo machine 10 of FIGS. 1A and 1B is shown in the flowcharts of FIGS. 2A-2E.

STEP 1: Apply power, by the power supply, to the negative terminal 22 and the positive terminal 24 of the support structure 12.

STEP 2: Energize the first and the second electromagnetic coils 18 and 20, respectively, to form an electromagnetic field.

STEP 3: Pull, by the electromagnetic field, the armature 56 down toward the first and the second electromagnetic coils 18 and 20, respectively.

STEP 4: Push, by the armature 56, the needle bar 44 and the tattoo needle 50 in a downward motion as a down stroke.

STEP 5: Bend the spring 26/28 downward in a direction towards the first and the second electromagnetic coils 18 and 20, respectively, since the armature 56 is connected to the spring 26/28.

STEP 6: Lose contact, by the angularly extending portion 104 of the spring 26/28, with the conductive contact screw 34 as the spring 26/28 bends downward.

STEP 7: Open the circuit between the angularly extending portion 104 of spring 26/28 and the conductive contact screw 34.

STEP 8: De-energize the first and the second electromagnetic coil 18 and 20, respectively, so as to form a de-energized first and a de-energized second electromagnetic coil 18 and 20.

STEP 9: Collapse the electromagnetic field formed by the de-energized first and the de-energized second electromagnetic coils 18 and 20, respectively.

STEP 10: Return the sprint 26/28 to its rest position.

STEP 11: Pull, by the spring 26.28, the armature 56, the needle bar 44, and the tattoo needle upward in an up stroke.

STEP 12: Complete, by the needle bar 44 and the tattoo needle, and entire needle stroke or one cycle.

STEP 13: Contact again the angularly extending portion 104 of the spring 26/28 with the conductive screw 34, since the spring 26/28 is now in its rest position.

STEP 14: Close again the circuit.

STEP 15: Return to STEP 1.

In the tattoo machine 10 of FIGS. 1A and 1B, the only way to control the operating parameters thereof is by mechanical adjustments. For example, by increasing the angle of the angularly extending portion 104 of the spring 26/28, the first and second electromagnetic coils 18 and 20, respectively, will cause the armature bar 56 to be closer to coils at which the same voltage will increase the frequency of the cycles, the force and the speed of the needle bar 44.

Experienced tattooists are able to implant an intended amount of ink to produce an intended effect by operating a tattoo machine running between 60 to 120 cycles per send, a stroke length between 2 and 3 millimeters and a force of approximately half a pound which is tested by feeling the resistance of the armature while running by placing a thumb against the armature bar 56. A typical setting is approximately 100 cycles, 2.5 millimeters and half a pound of pressure. Adjusting any variable mechanically affects the other parameters so it is difficult and arduous to adjust a machine optically by trial and error.

Additionally, in order for the conventional tattoo machine to work smoothly, it must be carefully tuned by further mechanical adjustments. A capacitor must be included to inhibit arcing of the contact points. The contact points are often difficult to adjust and maintain in proper mechanical attunement, and the capacitor in the device periodically fails. Additionally, in the conventional tattoo machine, operating parameters including frequency, i.e., number of needle strokes per unit of time, is difficult to control and alter with any precision because current affects both speed and magnetic strength of the coils simultaneously.

Thus, there exists a need to independently control the operating parameters of the stroke of a needle assembly of a tattoo machine.

Although this basic design has been used successfully for many years, in practice it does have several problems. Since the coils form an inductive circuit, the points arc when they open. The use of solid-state switches obviates the need to have mechanical contact points. Another significant problem is that it is difficult to adjust the machine mechanically so that it functions optimally. Generally, it is necessary to bend the spring and adjust the height of the contact point. This makes it difficult to get the mechanical adjustments just right. In addition, it is very difficult to alter the frequency of a mechanically switched machine.

### SUMMARY

One object of the embodiments of the present invention is to provide an astable multivibrator-triggered monostable multivibrator control device for a needle assembly of a tattoo machine, and for allowing an operator to selectively control frequency, length and force of a stroke of the needle assembly of the tattoo machine.

Another object of the embodiments of the present invention is to provide an astable multivibrator-triggered monostable multivibrator control device for a needle assembly of the tattoo machine, and for allowing an operator to selective control frequency, length, and force of a stroke of the needle assembly of the tattoo machine. The example control device includes an astable multivibrator and a monostable multivibrator. The astable multivibrator allows the operator to selectively control the frequency of the stroke of the needle assembly of the tattoo machine. The monostable multivibrator is triggered by the astable multivibrator and allows the operator to selectively control the pulse width of each cycle of the tattoo machine.

### BRIEF DESCRIPTION OF THE DRAWINGS

FIGS. 1A and 1B show views of a conventional tattoo machine at rest.

FIGS. 2A-2E show the operation of the tattoo machine of FIGS. 1A and 1B.

FIG. 3 shows waveforms in accordance with an example embodiment of the present invention.

FIG. 4 shows a schematic diagram of an example control device in accordance with the present invention.

### DETAILED DESCRIPTION OF EXAMPLE EMBODIMENTS

Electronic switching of the example embodiments of the present invention provides ease and flexibility while tuning the machine. There are several parameters that should be considered for proper machine functioning:

The stroke length should be long enough for the needles to move from being retracted into the tube where the needles can be coated with ink to being extended far enough to puncture the skin sufficiently to the desired depth.

Stroke frequency is difficult to alter on a mechanically switched machine. Frequency can be can be controlled with electronic circuitry that gates an electronic switch. Each machine tends to have an optimal frequency.

The actual stroke force that is delivered to the needle is a complex function of several variables, including, for example:
how far the spring holds the armature above the coils in the resting position;
spring stiffness;
power delivered to the coil, which is a function of voltage, frequency, and duty cycle;
stroke length;
bounce off the poles of the electomagnets;
friction of the moving parts of the machine;
resistance of the tattooee's skin to be punctured by different needle configuration; and
the number of needles in the cluster that penetrates the skin.

According to an embodiment of the present invention, a conventional tattoo machine can be adapted for use with the example control device, as will be described in further detail below.

According to example embodiments of the present invention, there are three electrical adjustments that can be made to achieve optimal performance for a particular effect that the tattoo artist wants to achieve, including:
voltage is control by an external variable DC power supply;
frequency, i.e., strokes per second, is controlled by an astable multivibrator. The astable multivibrator generates different pulse periods by adjusting a potentiometer that changes the resistance of the astable multivibrator's timing circuit. An astable multivibrator is an electronic circuit that switches rapidly between a high state and a low state. Thus, it outputs a rectangular wave.
duty cycle is the percentage of time that the electronic circuit is on during each stroke. A monostable circuit, i.e., a one-shot, uses a potentiometer to control the percentage of time the one-shot's output is on during each cycle. The duty cycle D is defined as the ratio between the pulse duration τ and the period T of a rectangular waveform.

D=τ/T

where:
τ is the duration that the function is non-zero
T is the period of the function.

In accordance with the present invention, the frequency and duty cycles are varied independently of the power supply voltage.

Tattoo machines tend to have a sweet spot where the operate optimally. Tuning a machine is a matter of finding the correct balance among the variables discussed above. Often it is difficult to find this balance using mechanical switching. Optimization, however, may be readily obtained with the electronic controls of the embodiments of the present invention.

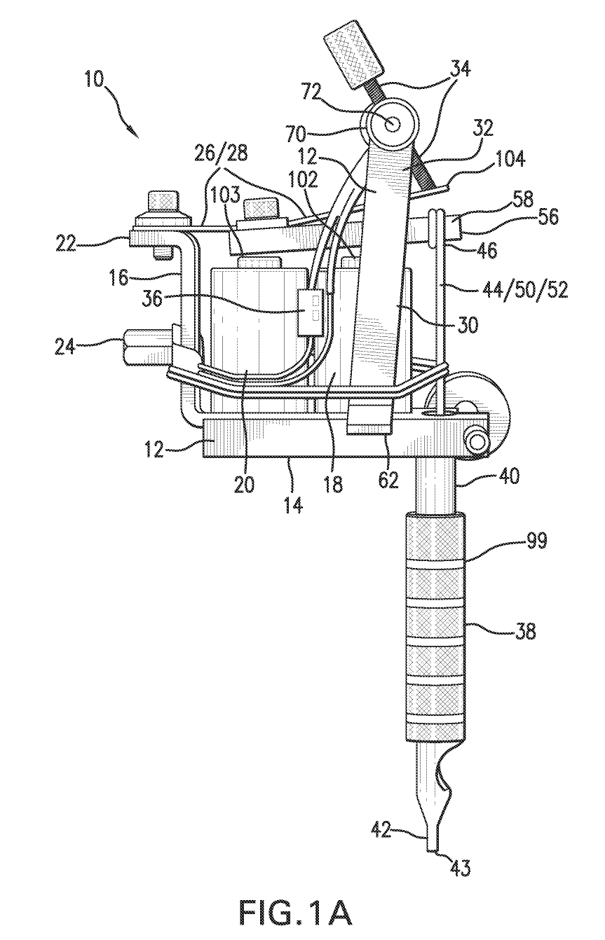

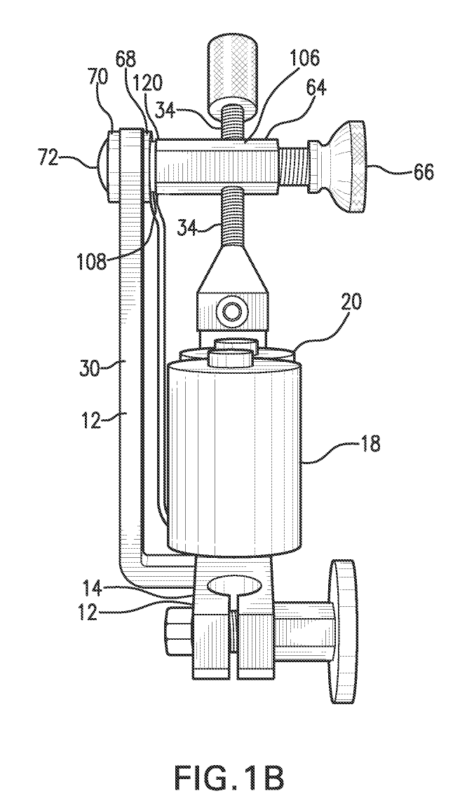

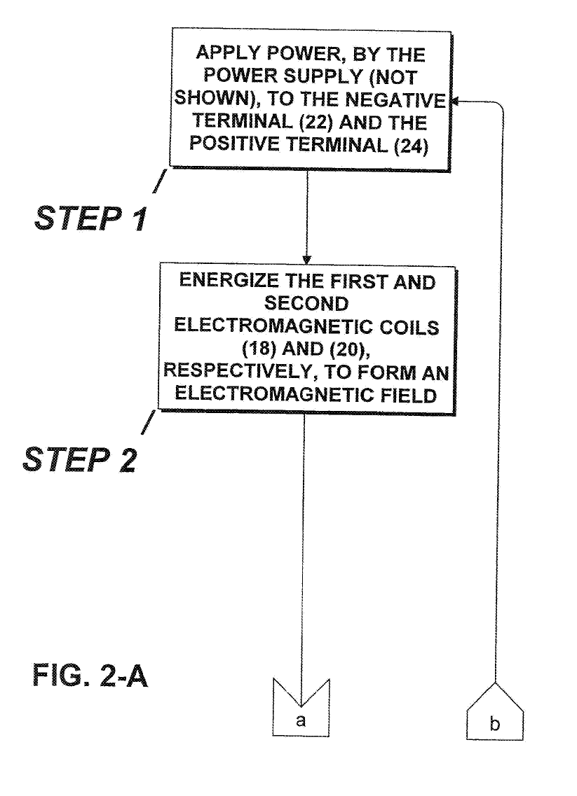

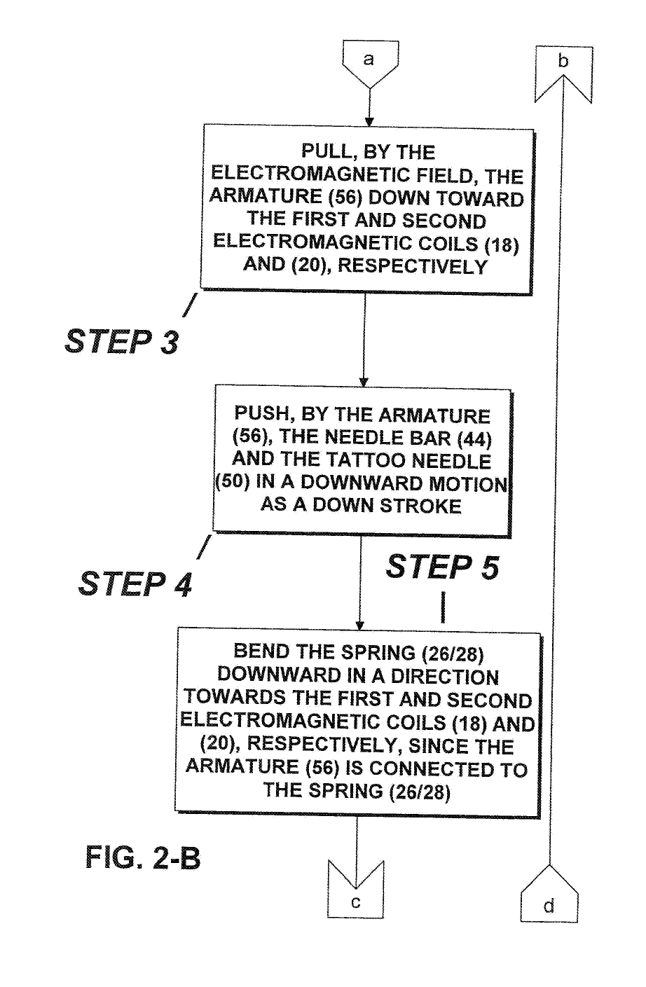

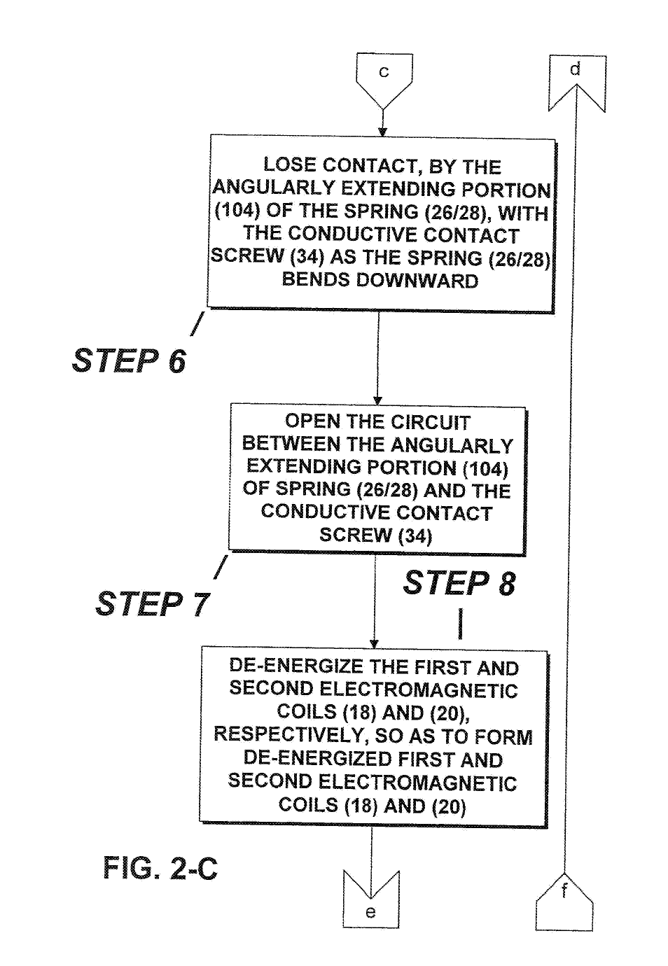

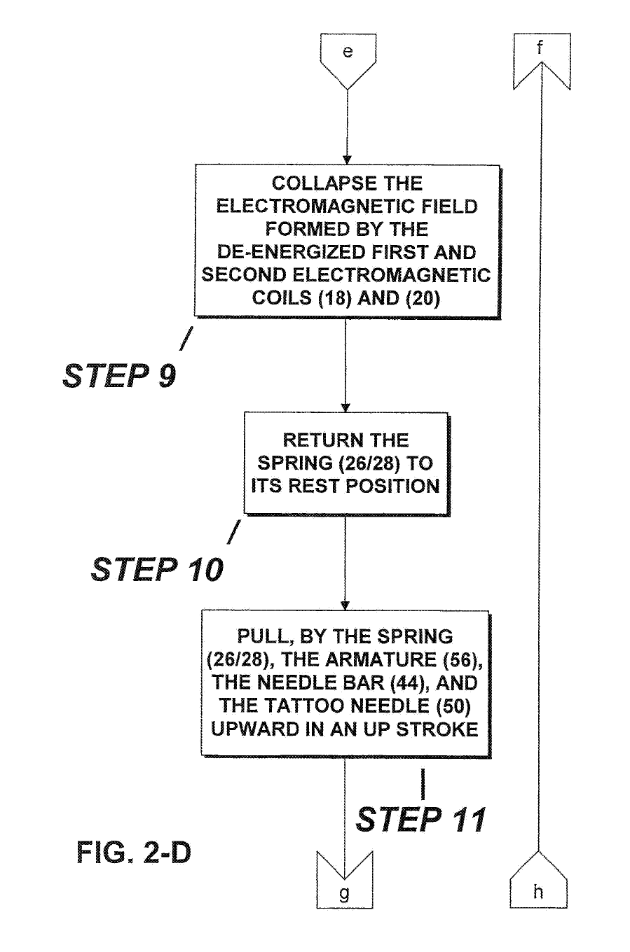

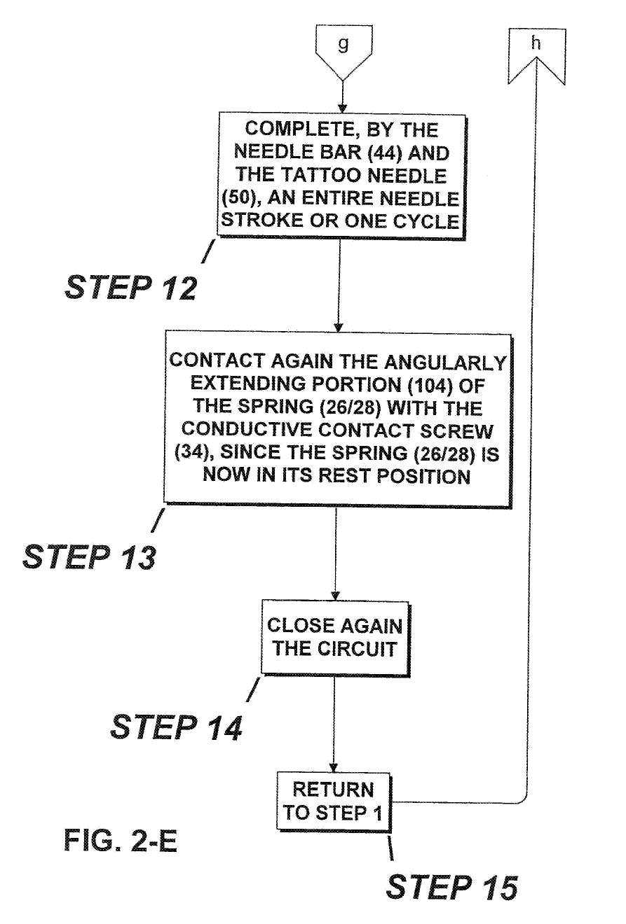

Modified tattoo machine: As noted above, a conventional tattoo device such as that illustrated in FIGS. 1A and 1B can be modified for use in accordance with the control device according to the present invention. For example, the capacitor 36 of the tattoo machine 10 may be cut off.

Additionally, the non-conductive recessed plastic washer 70 may be replaced with an electrically conducting washer so that the first coil 18 will be connected to the frame 12 which acts as the negative terminal at point 22 forming a closed circuit.

Control device: The control device may be connected (either permanently or releasably) to the positive terminal 24 and negative terminal 22 of the modified tattoo machine. The control device generates a square or pulsed signal that is applied to the positive and negative terminals 24 and 22. Although the signal is described herein as a “square wave,” the pulses of the signal may of course have a different shape, such as, rounded, triangular, trapezoidal, etc. The wave is referred to herein as a “square wave” for convenience, although the embodiments of the present invention are not so limited. The control device allows a user to independently control the frequency and duty cycle of the square wave, thereby controlling the needle stroke frequency, the needle stroke length, and the stroke force.

When the ON portion of the square wave is applied to the positive and negative terminals 24 and 22, respectively, the first and second electromagnetic coils 18 and 20, respectively, are energized, thus creating an electromagnetic field. The electromagnetic field pulls the armature 56 downward toward the first and the second electromagnetic coils 18 and 20, respectively, bending the spring 26. The armature 56 pushes the needle bar 44 and the tattoo needle downward in a down stroke, with the tattoo needle 50 penetrating the skin of the person being tattooed when the tattoo needle moves downwardly.

When the OFF position of the square wave is applied to the positive and negative terminals 24 and 22, respectively, the first and the second electromagnetic coils 18 and 20, respectively, de-energize, thus collapsing the electromagnetic field, thus causing the spring 26 to return to its rest position, pulling the armature 56, the needle bar 44, and the tattoo needle 50 upward with it in an upstroke. The tattoo needle is extracted from the skin of the person being tattooed during the upstroke.

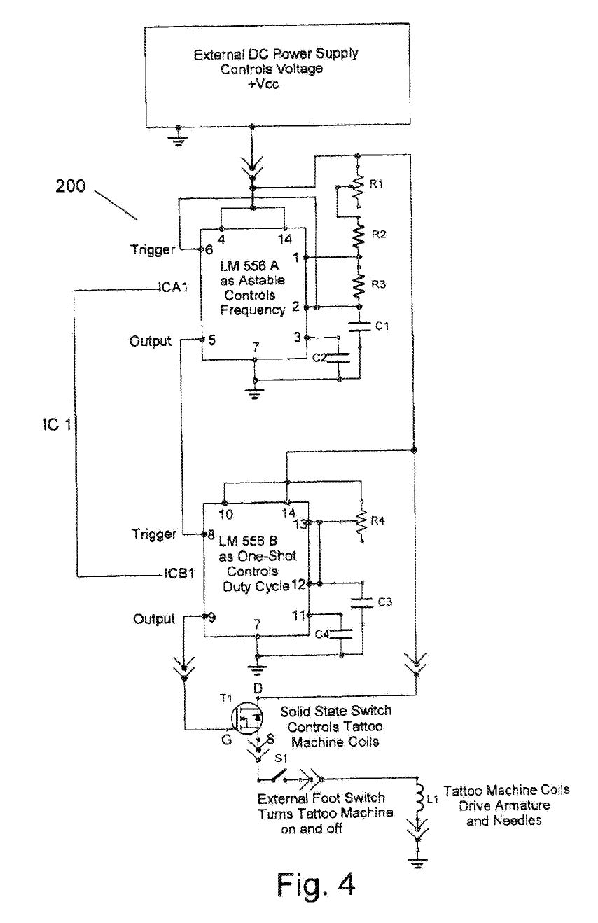

FIG. 4 shows the configuration of an example control device according to the present invention. The control device 200 includes an integrated circuit IC1 (which includes ICA1 and ICB1) and a ground GR. In this embodiment, IC1 is a National Semiconductor LM 556 dual timer. In accordance with this example embodiment, the ICA1 portion of integrated circuit IC1 is used to form an astable multivibrator to control frequency. The ICB1 portion of integrated circuit IC1 is used for form a monostable, i.e., a one-shot. In accordance with example embodiment, the one-shot is used to control the duty cycle.

Generally, a multivibrator is an electronic circuit used to implement a variety of simple two-state systems, such as oscillators, timers, and flip-flops. It is typically characterized by two amplifying devices, such as transistors, electron tubes, or other devices, cross-coupled by resistors and capacitors. The most common form is the astable or oscillating type, which generates a square wave. The high level of harmonics in its output is what gives the multivibrator its common name.

In an astable multivibrator circuit, the circuit is not stable in either state. It continuously oscillates from one state to the other. In a monostable multivibrator circuit, one of the states is stable, but the other is not. The circuit will flip into the unstable state for a determined period, and will return to the stable state after the determined time. This kind of a circuit is useful for creating a timing period of fixed duration in response to some external event. This circuit is also known as a one shot.

In its simplest form, the multivibrator circuit consists of two cross-coupled transistors. Using resistor-capacitor networks within the circuit to define the time periods of the unstable states, the various types may be implemented. Multivibrators find applications in a variety of systems where square waves or timed intervals are required.

The control device 200 includes a voltage source interface for interfacing the control device 200 with a positive voltage source +Vcc.

The control device 200 further comprises a first capacitor C1, a second capacitor C2, a third capacitor C3, and a fourth capacitor C4.

The control device 200 further includes a first resistor—potentiometer R1, a second resistor R2, a third resistor R3, and a fourth resistor—potentiometer R4.

The control device 200 further includes a MOSFET T1 having a gate G, a drain D, and a source S.

FIG. 4 also shows an inductor L1, which represents the first electromagnetic coil 18 and the second electromagnetic coil 20 of the tattoo machine shown in FIGS. 1A and 1B.

As shown in FIG. 4, the integrated chip IC1 comprises pins 1-14. The pins 1, 2, 3, 4, 5, 6, 7 and 14 are of the first portion ICA1 of the integrated chip IC₁ and are configured as an astable multivibrator that controls the frequency of the square wave. The pins 7, 8, 9, 10, 12,1 3 and 14 are of the second portion ICB1 of the integrated chip IC₁ and are configured as a monostable multivibrator that is triggered by the astable multivibrator and controls the duty cycle.

The pin 6 of the first portion ICA1 of the integrated chip IC1 is connected to the pin 2 of the first portion ICA1 of the integrated chip IC1.

The pin 7 of the first portion ICA1 of the integrated chip IC1 is connected to ground GR.

The output of the astable pin 5 is connected to pin 8 of the second portion of ICB1 of the integrated chip IC₁, which is the input of the monostable.

The pin 9 of the second potion ICB1 of the integrated chip IC₁ is connected to gate G of the MOSFET T1.

The pin 10 of the second portion ICB1 of the integrated chip IC1 is connected to the positive voltage source +Vcc, though the positive voltage source interface.

The fourth resistor—potentiometer R4 is connected to the positive voltage source +Vcc through the positive voltage source interface.

The pin 11 of the second portion ICB1 of the integrated chip IC₁ is connected to the fourth capacitor C4.

The second capacitor C2 is connected to the ground GR.

The pin 12 of the second portion ICB1, of the integrated chip IC1, is connected to the third capacitor C3.

The first capacitor C1 is connected to the ground GR.

The pin 12 of the second portion ICB1 of the integrated chip IC1 is connected in series to the fourth resistor—potentiometer R4 and to the pin 13 of the second portion ICB1 of the integrated chip IC1.

The pin 13 of the second portion ICB1 of the integrated chip IC₁ is connected to the wiper of the fourth resistor—potentiometer R4.

The first resistor—potentiometer R1 is connected to the positive voltage source +Vcc through the positive voltage source interface.

The pin 1 of the first portion ICA1 of the integrated chip IC₁ is connected to junction between the second resistor R₂ and the third resistor R₃.

The second resistor R₂ is connected to the first resistor—potentiometer R₁.

The pin 1 of the first portion ICA1 of the integrated chip IC1 is connected to the pin 2 of the first portion ICA1 of the integrated chip IC1 through the third resister R3.

The pin 2 of the first portion ICA1 of the integrated chip IC₁ is connected to the ground GR through the first capacitor C1.

The pin 2 of the first portion ICA1 of the integrated chip IC1 is connected to the first capacitor C1. The first capacitor C1 is connected to ground GR.

The pin 4 of the first portion ICA1 of the integrated chip IC1 is connected to the positive voltage source +Vcc through the positive voltage source interface.

The pin 7 of the first portion ICA1 of the integrated chip IC1 is connected to ground GR.

The drain D of the MOSFET T1 is connected to the positive voltage source +Vcc through the positive voltage source interface.

In this embodiment, the frequency of the square wave generated by the control device 200 is determined by the first resistor—potentiometer R1, the second resistor R2, the third resistor R3, and the first capacitor C1. The first resistor—potentiometer R1, allows adjustment of the frequency from approximately 65 HZ to approximately 120 Hz utilizing, for example, a knob.

The output of the monostable multivibrator controls the gate G of the MOSFET T1, that functions as an electronic switch. The MOSFET T1, powers the inductor L1. Adjustment of the positive voltage source +Vcc, e.g.,+Vcc+5 volts to 16 volts, gives further refinement of the control of the needle stroke frequency, stroke length, and stroke force.

The fourth resistor—potentiometer R4 and the third capacitor C3 control the length of time that the output is high, i.e., control the duty cycle. The fourth resistor -potentiometer R4 is adjusted by, for example, a knob to vary the duty cycle.

FIG. 4 also shows an external foot switch S1. The external foot switch S1 of the control device 200 is connected to the source S of the MOSFET T1 and is for selectively turning the tattoo machine ON and OFF, freeing the hands of the operator.

In this embodiment, the electronic implementation of the system described above includes:
an external variable voltage DC power supply of 5-18 volts supplying power to an LM556 integrated chip and to the drain of a MOSFET (in this embodiment, IRF510), wherein the LM556 is a dual timer;
one half of the LM556 is configured as a astable multivibrator, wherein potentiometer R1, resistors R2 and R3, and a capacitor C1 control the frequency of the astable multivibrator
the output of the astable multivibrator is connected to the input of the other half of the LM556, which is configured as a monostable multivibrator, i.e., a one-shot, wherein the one-shot is triggered by the negative edge of the pulse generated by the astable multivibrator, wherein the length of time that the one-shot remains in its high state is controlled by a variable potentiometer R4 and a capacitor C1, and wherein this controls the duty cycles;
the output of the one-shot is connected to the gate of the MOSFET that function as a solid-state switch;
the external mechanical switch, e.g., a foot switch, is connected to the output S of the solid-state switch MOSFET; and
the mechanical foot switch is connected to the coils of the tattoo machine wherein the foot switch turns the tattoo machine on and off.

The example embodiments of the present invention control the amplitude, frequency and duty cycle of the power to the tattoo machine. These three variable interact with the mechanical properties of the tattoo machine, i.e.,:
the static and dynamic properties of the spring;
the angle to which the spring is bent, distance of he armature above the poles of the electromagnets;
the adjustment of the screw 34 which limits stroke length; and
the friction of the system.

In may be helpful in the embodiments of the present invention to place a material that can absorb and reflect the force of the armature when the armature hits the poles. For example, a simple rubber band can be used to help the armature bounce upward at the end of each downward stroke. This may also prevent the armature from getting stuck in the downward position against the pole of the magnet, and it may significantly reduce the noise that the tattoo machine makes.

The duty cycle control of the embodiments of the present invention may strongly influence the stroke length and stroke force. The duty cycle may also have a great effect on the amount of current the coils draw. By varying the duty cycle by the embodiments of the present invention, the current can be adjusted to a minimum amount for effective operation because of their small size coils will heat up and lose force when the amperage exceeds ¾ of an amp.

The flexibility of the embodiments of the present invention make the process of tuning a tattoo device simple and straightforward. Alterations of the DC supply voltage to the IC1 (LM556) do not change the frequency or the on-time of the one-shot. Hence, voltage can be adjusted by the embodiments of the present invention independently of the other two variables. In the example embodiments, the frequency and the duty cycle interact. For a given on-time τ of the one-shot, the percentage of time that the one-shot is on, per period, increases as the frequency increased. Thus, the duty cycle changes with frequency. Nevertheless, it is still possible to change the frequency and duty cycle independently of each other by altering the on-time of the one-shot of the embodiments of the present invention at any given frequency.

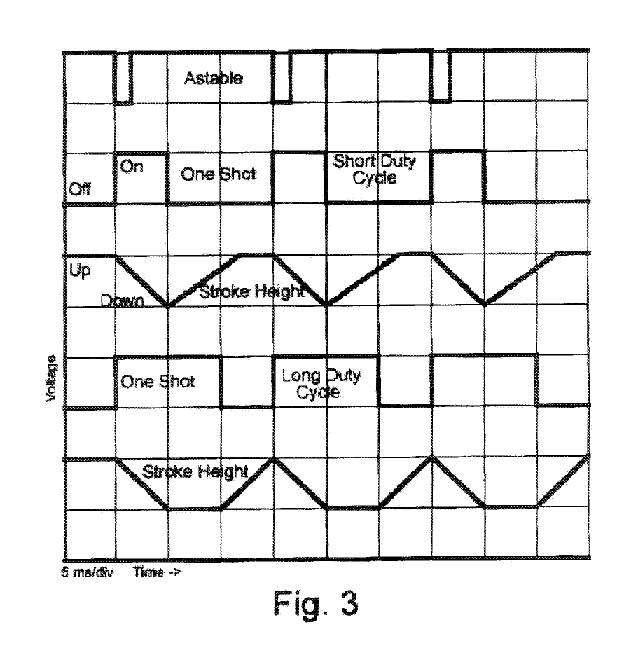

FIG. 3 is a diagrammatic representation of an example of different wave forms. Here, the astable multivibrator of the embodiments is running with a period of 15 ms, which is a frequency of 66.6 cycles per second. In the short duty cycle case, i.e., 33%, the on-time of the one-shot is 5 ms. In the long duty case, i.e., 66%, the on-time of the one-shot is set at 10 ms. In the short duty cycle case, the tattoo machine of the embodiments of the present invention is toward the top of its stroke more time than it is at the bottom of its stroke. Conversely, in the long duty cycle case, the stroke is at the bottom of its cycle longer than at the top. Which case would be better depends on the demands of the particular situation. In any case, adjustments are readily made by the embodiments of the present invention.

The independence of the three variables, voltage, frequency, and pulse width, simplifies tuning of the tattoo machine. To facilitate tuning the machine even more precisely, the stroke length can also be limited mechanically by adjusting the screw 34 that controls the upper limit of the movement of the armature 56. Limiting the stroke length mechanically allows the operator to vary the stroke frequency over a wide range. Lower frequencies allow longer stroke length. Higher frequencies generally require shorter lengths. The pulse width adjustment overcomes the many variables, i.e., spring tension, friction, etc, which affect the proper operation of the tattoo machine. This control enables the operator to find an optimal duty cycle for each frequency, which in turn, produces a consistently powerful stroke. Thus, the simple and precise electronic adjustment of the duty cycles overcomes the frustrating difficulties of trying to mechanically tune tattoo machines at various frequencies.

1. A control device for a tattoo device, the control device comprising:
an astable multivibrator adapted to selectively control a stroke frequency of a needle assembly of the tattoo device, wherein the tattoo device includes coils for driving the needle assembly; and
a monostable multivibrator, triggered by the the astable multivibrator, the monostrable multivibrator adapted to selectively control a length of time that the coils of the tattoo machine are turned ON during each stroke of the needle assembly of the tattoo device.

2. The control device according to claim 1, wherein the astable multivibrator and the monostable multivibrator are formed from at least one integrated chip.

3. The control device according to claim 1, further comprising:
at least one potentiometer, wherein the stroke frequency and the length of stroke are adjusted via the at least one potentiometer.

4. The control device according to claim 1, wherein the control device includes a least one user control via which the user adjusts the stroke frequency and the length of stroke.

5. A control device for a tattoo apparatus, the control device generating a pulsed signal for controlling a reciprocation of a needle assembly of a tattoo device, the control device including first circuitry for selectively adjusting a duty cycle of pulses of the pulsed signal.

6. The control device according to claim 5, wherein the control device further includes second circuitry for selective adjusting a frequency of the pulsed signal.

7. The control device according to claim 6, wherein the frequency and the duty cycle are adjustable by a user.

8. The control device according to claim 5, wherein the duty cycle controls a stroke length of the needle assembly.

9. The control device according to claim 6, wherein the frequency of the pulsed signal controls a stroke frequency of the needle assembly.

10. A tattoo system, comprising:
a reciprocable tattoo needle assembly which can limit stroke length; and
a control device adapted to generate a pulsed signal for controlling a reciprocation of the needle assembly, the control device including first circuitry for selectively adjusting a duty cycle of pulses of the pulsed signal.

11. The tattoo system according to claim 10, wherein the control device further includes second circuitry for selective adjusting a frequency of the pulsed signal.

12. The tattoo system according to claim 11, wherein the frequency and the duty cycle are adjustable by a user.

13. The tattoo system according to claim 10, wherein the duty cycle controls the stroke length of the needle assembly.

14. The tattoo system according to claim 11, wherein the frequency of the pulsed signal controls a stroke frequency of the needle assembly.
